(19) United States
(12) Patent Application Publication
(10) Pub. No.: US 2023/0055165 A1
(43) Pub. Date: Feb. 23, 2023

(54) Dynamic Wearable Tightness Suggestions

(71) Applicant: Google LLC, Mountain View, CA (US)

(72) Inventor: Jonathan D. Hurwitz, San Jose, CA (US)

(21) Appl. No.: 17/405,537

(22) Filed: Aug. 18, 2021

(51) Int. Cl. A61B 5/024; A61B 5/00; A61B 5/0205; G06N 20/00

(52) U.S. Cl. CPC A61B 5/02416; A61B 5/681; A61B 5/02055; G06N 20/00; A61B 5/7267; A61B 5/742

(57) ABSTRACT

A trained model running on a wearable device can be used to analyze PPG inputs and error/confidence levels, internal and/or external temperature inputs, and motion sensor data to determine whether the wearable device can be more tightly secured to the body of a user to improve PPG or other health sensor signals. A clustering model can be used to analyze data in real time or close to real time, and provide a notification to the user. The notifications can indicate steps to be taken to improve the signal quality from the wearable device, such as tightening the device.

### BACKGROUND

Photoplethysmogrpahy (PPG) is an optical technique used to detect changes in circulation. PPG has become ubiquitous in wearable devices for measuring heart rate or detecting other information related to a user wearing the wearable device.

In colder temperatures, vessels that supply blood to surface tissues will constrict to preserve body heat and preserve an individual's core temperature. This process is known as vasoconstriction. As PPG relies on optically “reading” or detecting changes in blood vessels that are relatively close to the skin of a user, such as those on the top of the wrist, vasoconstriction in cold temperatures can affect the measurements by decreasing the quality of usable signal. This effect is exacerbated by users who do not have their watch tight enough for the sensor cluster to measure the optical signal.

### SUMMARY

Aspects of the present disclosure include methods, systems, and apparatuses for providing dynamic tightness suggestions on a user device. The tightness suggestions can suggest actions that a user can take with respect to the user device to enhance detection aspects of the user device, such as for example, PPG readings.

A high quality PPG measurement relies on a high signal to noise ratio (SNR). Example factors which can influence PPG include (i) the optical design of a user device, including the layout of the LEDs and photodiodes as well the design of the optical window; (ii) the selection of electronics and optical components; and (iii) the degree to which a PPG sensor or PPG module is depressed into or proximate to a user's skin to minimize the amount of light artifacts introduced into the PPG sensor or module, which erode or introduce noise into the PPG signal, particularly during high motion activities.

Aspects of the present disclosure include any combination of the following features, whether found in methods, systems, non-transitory computer readable media, or apparatuses.

Aspects of the present disclosure include a method for providing information related to fit of a wearable device, The method can comprise receiving external temperature sensor data from one or more sensors configured to obtain an external temperature of the wearable device; receiving health data from one or more health sensors of the wearable device; analyzing, using a trained machine learning model, metrics related to the external temperature sensor data and the health data, to obtain an output; determining, from the output, whether to suggest adjusting the fit of the wearable device; and generating a notification based on the determining. The method can include comprising receiving internal temperature sensor data from one or more sensors configured to obtain temperature from inside the wearable device. A temperature offset can be applied to the external temperature sensor data based on the measured internal temperature sensor data. A change in the fit can be detected upon a user taking an action on the user device. The method can comprise comparing a second output to the first output, wherein the second output is obtained by analyzing, using a trained machine learning model, metrics obtained after detection of a change in the fit. The method can comprise updating or removing the notification when the second output sufficiently differs from the first output. The trained model can be a clustering model. The health sensor can be a PPG sensor. The one or more sensors can comprise a gyroscope or accelerometer. The method can comprise receiving motion sensor data from one or more motion sensors of the wearable device and wherein the analyzing analyzes metrics related to the motion sensor data. The motion sensor data can be analyzed to determine a bobbing motion of the wearable device. Additional external temperature data can be obtained from a second wearable device. Health data can be raw or processed data from which photoplethysmography can be performed.

Aspects of the disclosed technology can include a wearable device. The wearable device can comprise a communications interface; a display; and one or more computing devices coupled to one or more memory devices. The one or more memory devices containing instructions that cause the one or more computing devices to receive external temperature sensor data from one or more sensors configured to obtain an external temperature of the wearable device; receive health data from one or more health sensors of the wearable device; receive motion sensor data from one or more motion sensors of the wearable device; analyze, using a trained machine learning model, metrics related to the external temperature sensor data and, the health data, and the motion sensor data, to obtain an output; determine, from the output, whether to suggest adjusting the fit of the wearable device; and generate a notification based on the determining. The instructions can be configured to receive internal temperature sensor data from one or more sensors configured to obtain temperature from inside the wearable device. A temperature offset can be applied to the external temperature sensor data based on the measured internal temperature sensor data. The instructions can be configured to detect a change in the fit upon a user taking an action on the user device. The instructions can be configured to compare a second output to the first output, wherein the second output is obtained by analyzing, using a trained machine learning model, metrics obtained after detection of a change in the fit. The instructions can be configured to receive motion sensor data from one or more motion sensors of the wearable device and wherein the analyzing analyzes metrics related to the motion sensor data.

Aspects of the disclosed technology can include a non-transient computer readable medium containing program instructions, the instructions when executed perform the steps of: receiving external temperature sensor data from one or more sensors configured to obtain an external temperature of a wearable device; receiving health data from one or more health sensors of the wearable device; analyzing, using a trained machine learning model, metrics related to the external temperature sensor data and the health data, to obtain an output; determining, from the output, whether to suggest adjusting the fit of the wearable device; and generating a notification based on the determining.

### BRIEF DESCRIPTION OF THE DRAWINGS

The accompanying drawings are not intended to be drawn to scale. Like reference numbers and designations in the various drawings indicate like elements. For purposes of clarity, not every component may be labeled in every drawing. In the drawings:

FIG. 1 is an example schematic drawing of electronics according to aspects of this disclosure.

FIG. 2A is an example illustration of a wearable user device according to aspects of this disclosure.

FIG. 2B is a diagram of an example cross section of a user device on a user's skin according to aspects of this disclosure.

FIG. 2C is an example schematic diagram of communication between devices according to aspects of this disclosure.

FIG. 3 is a block diagram illustrating an example architecture according to aspects of this disclosure.

FIG. 4 is a flowchart of an example method according to aspects of this disclosure.

### DETAILED DESCRIPTION

### Overview

This technology relates to identifying when a user device can be tightened or adjusted to improve data or signals obtained by the device and generating or providing notifications to a user on a user device for the same. For example, the technology can detect when a user device, such as a smartwatch, can be more closely secured to a user.

The device can identify when conditions, such as cold weather, are present which can degrade health signals obtained from the user based on temperatures from (i) an external, externally oriented sensor, or “skin temperature” sensor, and (ii) a sensor internal to a user device or internally oriented to detect the temperature inside the device. For example, based on a low temperature recorded by both the internal sensor and the external sensor, it can be determined that a cold weather condition exists. In other examples, the internally oriented sensor can be used to adjust the readings obtained from the externally oriented sensor when the internal sensor records a higher temperature than the external temperature.

The device can further identify the “tightness” or “looseness” of a user device being placed on a user, such as for example, a smartwatch or other wearable on a user's wrist. In some examples, the “tightness” can be determined based on motion sensor data and optical data being obtained by the user device. For instance, if there is a threshold degree of optical noise from ambient light and a threshold amount of movement or “bobbing” of the user device being detected by a motion sensor, it can be determined that the smartwatch is loosely secured to the user's wrist and that adjusting the watch band to be secured more tightly may result in improved performance of the smartwatch with respect to measurements, health tracking, and the like. In some examples, the “confidence levels” or quality of signals can be estimated for various signals, and these confidence levels or quality of signals can be used in analysis.

In some examples, the device can identify states of the device, such as fitted too loosely, fitted too tightly, fitted properly, etc., based on multiple inputs which are used by a trained machine learning model to infer the states. The outcome from the trained machine learning model can be used to determine one or more notifications, messages, or instructions to display to a user which can be implemented by the user to improve the quality of signals from the user device.

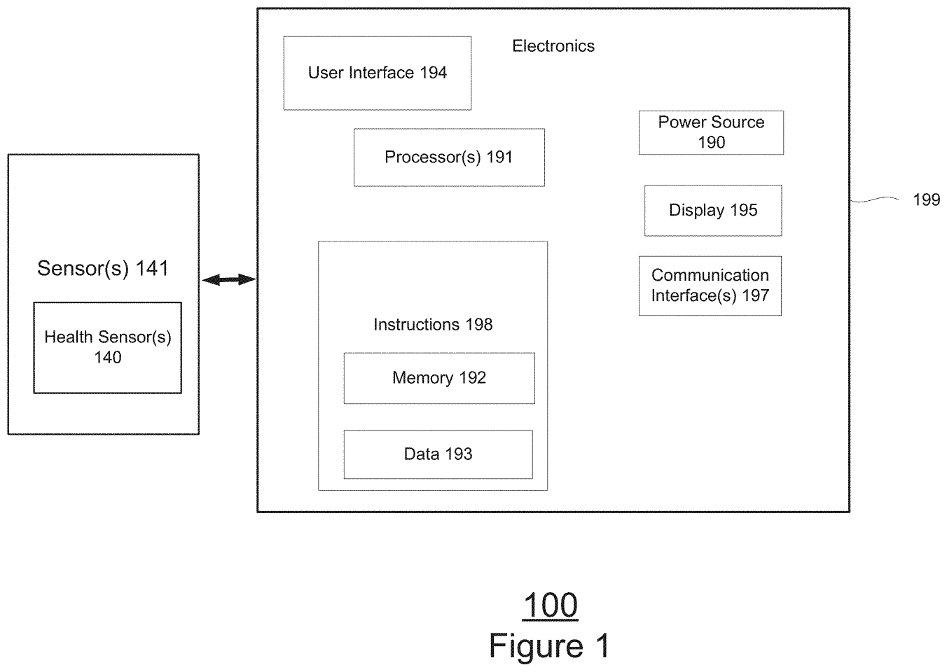

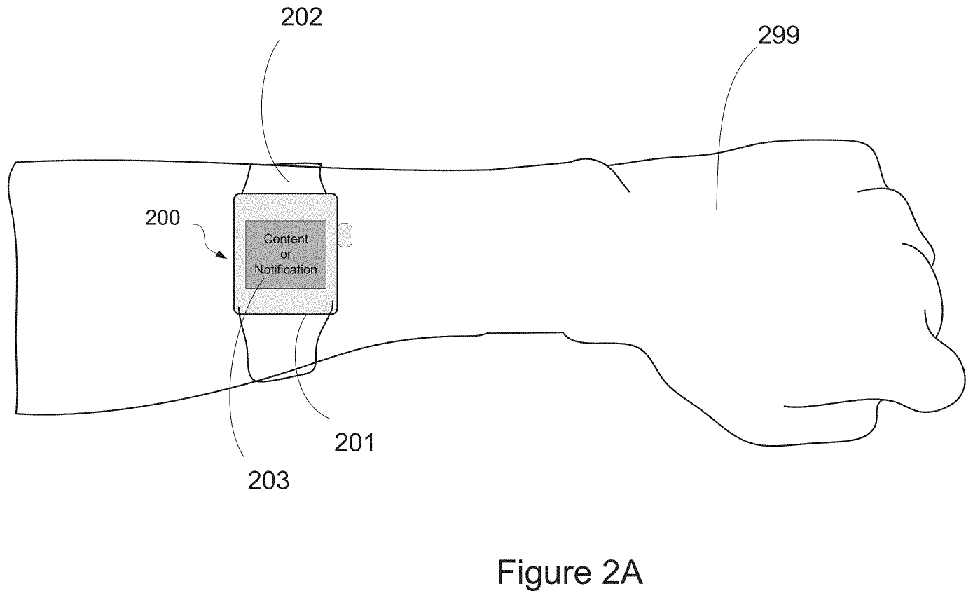

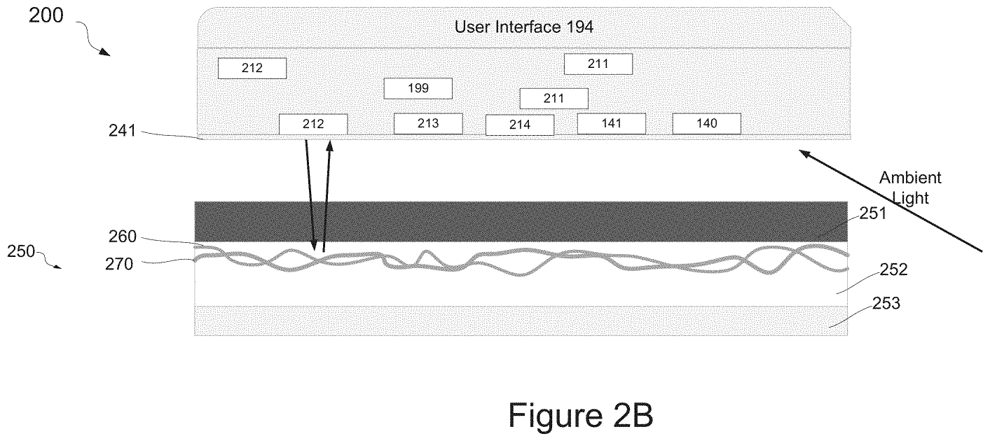

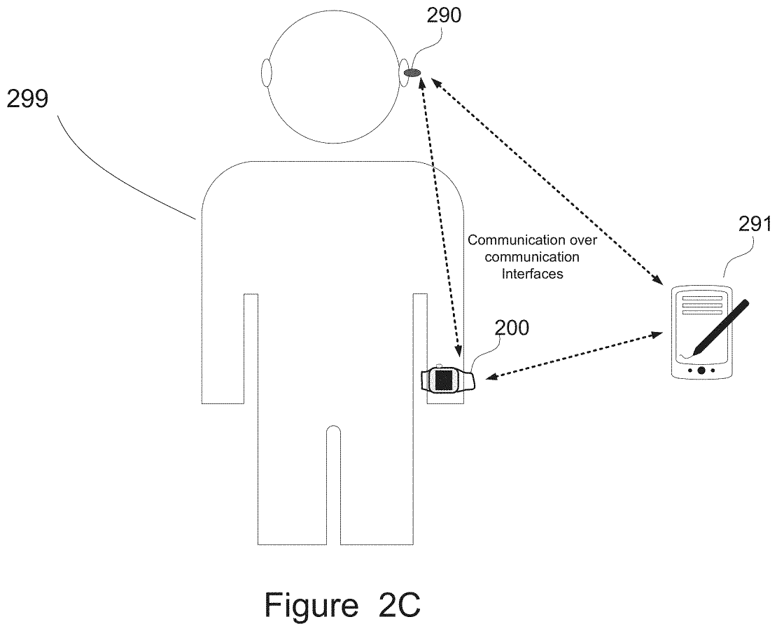

FIG. 1 illustrates an example system 100. Example system 100 can be included within a user device, such as for example, user device 200 described with respect to FIG. 2. As explained below, example system 100 can include multiple sensors and processing units to obtain user related information and device information. In broad detail, example system 100 can contain electronics 199, further described below, and multiple sensors, such as health sensor(s) 140 and sensor(s) 141. Example system 100 can, based on data obtained by multiple sensors, be used to send a notification to a user to adjust the fit of a user device when it is determined that tightening or adjusting the fit of the user device will improve the quality of health related information being obtained from the device.

Illustrated in FIG. 1 is a bidirectional arrow indicating communication(s) between electronics 199, health sensor(s) 140, and sensor(s) 141. Health sensor(s) 140 can be a subset of sensor(s) 141. Each sensor can also be used to obtain confidence values or other statistical information related to the sensed data, such as for example, sampling rate or precision. Health sensor(s) 140 may be configured to obtain information related to the health or physical condition of a user while sensor(s) 141 may be configured to determine information related to a user device, such as tilt, motion, acceleration, internal temperature of the device, or external temperature of the device. Data from health sensor(s) 140 can be analyzed to determine a physical condition or health condition of a user, such as blood pressure, heart rate, blood oxygen level, stress, or atrial fibrillation. Data from sensor(s) 141 can be analyzed to interpret information related to a user device, such as whether the device is “bobbing,” other secondary motion of the device such as a wobble, the internal or external temperature of the device, relative motion of the device compared to a user, and acceleration and velocity data of the device. Although health sensor(s) 140 and sensor(s) 141 are illustrated separately, in some examples, information from either can be used to analyze both health information and device information.

FIG. 1 illustrates aspects of electronics 199, which may be used in aspects of the disclosed technology as described in further detail below. Electronics 199 can be included within or be any computing device which is capable of performing the steps and algorithms described herein, such as without limitations, cell-phones, tablets, computers, laptops, servers, smart devices, wearable devices, or smart watches. Although the description in FIG. 1 is given with respect to electronics 199, a person of skill in the art should understand that in some examples electronics 199 can be combined or operate collectively with health sensor(s) 140 and sensor(s) 141.

Health sensor 140 can be any device, circuitry, or module which can be used to observe or determine information related to a health state of a user, such as, for example, blood pressure, blood oxygen levels, stress, temperature, or other metrics which can be derived from a combination of the exemplary aforementioned metrics. Non-limiting examples of health sensor 140 includes PPG sensors or modules, temperature sensors, infra-red sensors, photodiodes, and the like. Health sensor 140 can be a digital or analog sensor. Health sensor 140 can contain additional components such as an analog front end, photodetectors, accelerometers, or health sensors, such as photoplethysmography sensors, devices, or circuitry. In some examples, a health sensor need not be part of the same device as electronics 199, and can be included in a separate device. Other arrangements of components described with respect to FIG. 1 are within the scope of this disclosure. In other examples, health sensor 140 can be included or arranged within user devices, such as a mechanical watch, a smart watch, a smart ring, a cell phone, earbud, headphone, armband, fitness tracker, smart clothing, laptop computer, or any other electronic device including but not limited to wearable electronic devices. In other examples, health sensor 140 can be integrated into jewelry, such as a pendant, necklace, bangle, earring, armband, ring, anklet, or other jewelry.

Sensor(s) 141 can be any device, circuitry, or module which can obtain information related to the environment or related to a user device. For example, sensor 141 can be a temperature sensor, proximity sensor, accelerometer, infrared sensor, pressure sensor, light sensor, touch sensor, humidity or sweat detection sensor, gyroscope, magnetic sensors, microphones, or tilt sensor. As described with respect to health sensor(s) 140, sensor(s) 141 may be included within a user device depending on the device. As further explained herein, sensor(s) 141 may be used to detect an “internal” temperature sensor and an “external” temperature sensor.

Electronics 199 may contain a power source 190, processor(s) 191, memory 192, data 193, a user interface 194, a display 195, communication interface(s) 197, and instructions 198. The power source may be any suitable power source to generate electricity, such as a battery, a chemical cell, a capacitor, a solar panel, or an inductive charger. Processor(s) 191 may be any conventional processors, such as commercially available microprocessors or application-specific integrated circuits (ASICs). Memory may store information that is accessible by the processors, including instructions that may be executed by the processors and data. Memory 192 may be a type of memory operative to store information accessible by the processors, including a non-transitory computer-readable medium, or other medium that stores data that may be read with the aid of an electronic device, such as a hard-drive, memory card, read-only memory (“ROM”), random access memory (“RAM”), optical disks, as well as other write-capable and read-only memories. The subject matter disclosed herein may include different combinations of the foregoing, whereby different portions of the instructions and data are stored on different types of media. Data 193 of electronics 199 may be retrieved, stored or modified by the processors in accordance with the instructions 198. For instance, although the present disclosure is not limited by a particular data structure, data 193 may be stored in computer registers, in a relational database as a table having a plurality of different fields and records, XML documents, or flat files. Data 193 may also be formatted in a computer-readable format such as, but not limited to, binary values, ASCII or Unicode. Moreover, data 193 may comprise information sufficient to identify the relevant information, such as numbers, descriptive text, proprietary codes, pointers, references to data stored in other memories (including other network locations) or information that is used by a function to calculate the relevant data.

Instructions 198 may control various components and functions of health sensor(s) 140 or sensor(s) 141. For example, instructions 198 may be executed to selectively obtain or analyze information from a PPG sensor, a motion sensor, and an ambient light sensor to determine if a user device is properly secured or attached to a user. In some examples, algorithms can be included as a subset of or otherwise as part of instructions 198 included in electronics 199.

Instructions 198 can include algorithms to interpret or process information received from the health sensor(s) 140, sensor(s) 141, or from other parts of electronics 199. For example, instructions 198 can analyze information received through or generated by analyzing health information from health sensor 140, information in data 193, information displayed on display 195, or information processed by processor(s) 191.

For example, physical parameters of the user can be extracted or analyzed through algorithms. Without limitation the algorithms could use any or all information about the waveform, such as shape, frequency, or period of a wave, Fourier analysis of data or a signal obtained from health sensor 140, harmonic analysis, pulse width, pulse area, peak to peak interval, pulse interval, intensity or amount of light received by a photodetector, wavelength shift, or derivatives of the signal generated or received by health sensor 140. Other algorithms can be included to calculate absorption of oxygen in oxyhemoglobin and deoxyhemoglobin, heart arrhythmias, heart rate, premature ventricular contractions, missed beats, systolic and diastolic peaks, and large artery stiffness index. In yet other examples, artificial learning or machine learning algorithms can be used in both deterministic and non-deterministic ways to extract information related to a physical condition of a user such as blood pressure and stress levels, from, for example, heart rate variability. PPG can also be used to measure blood pressure by computing the pulse wave velocity between two points on the skin separated by a certain distance. Pulse wave velocity is proportional to blood pressure and that relationship can be used to calculate the blood pressure. In some examples, the algorithms can be modified or use information input by a user into memory of electronics 199 such as the user's weight, height, age, cholesterol, genetic information, body fat percentage, or other physical parameters. In other examples, machine learning algorithms can be used to detect and monitor for known or undetected health conditions, such as an arrhythmia, based on information generated by the photodetectors, health sensors, and/or processors.

In some examples, instructions 198 can also analyze information related to an operating condition or environment in which a user device is operating. For instance, data obtained from sensor(s) 141, such as from a gyroscope or motion sensor, can be analyzed for “bobbing” or other types of “secondary” motion. In other examples, instructions 198 can also include trained or untrained machine learning models as described herein.

User interface 194 may be a screen which allows a user to interact with health sensor 140, such as a touch screen or buttons. Display 195 can be an LCD, LED, mobile phone display, electronic ink, or other display to display information about health sensor 140. User interface 194 can allow for both input from a user and output to a user. Examples of such output include notifications to the user suggesting that a fit of the device on the user's body should be adjusted to obtain more accurate device performance. For example, the notification may be a message displayed on a screen indicating that the device is too loosely secured and/or that the device should be tightened. In some examples, the user interface 194 can be part of electronics 199, while in other examples, the user interface can be considered part of a user device. User interface 194 may also comprise devices such as keyboards, etc.

Communication interface(s) 197 can include hardware and software to enable communication of data over standards such as Wi-Fi, Bluetooth, infrared, radio-wave, and/or other analog and digital communication standards. Communication interface(s) 197 allow for electronics 199 to be updated and information generated by health sensor 140 to be shared to other devices. In some examples, communication interface(s) 197 can send historical information stored in memory 192 to another user device for display, storage, or further analysis. In other examples, communication interface(s) 197 can send the signal generated by the photodetector to another user device in real-time or afterwards for display on that device. Communication interface(s) 197 can include Bluetooth, Wi-Fi, Gazelle, ANT, LTE, WCDMA, or other wireless protocols and hardware which enable communication between two devices.

FIG. 2A illustrates a user device, 200, which can be worn by a user, such as user 299. User device 200 can display a notification to user 299 to tighten or adjust user device 200 on the user's body when it is determined that a tighter or adjusted fit would give a more accurate reading or improve the quality of signals being obtained by user device 200.

User device 200 can include a housing 201, and a strap 202. Housing 201 can have components such as a back portion, which will contact the skin of user 299. The back portion can contain a glass portion which will allow light to pass through the back portion. For example, light can be generated from other components contained within housing 201, such as a light source. User device 200 and housing 201 can also have a user interface which allows user 299 to interact and view information from user device 200. The user interface can be part of a touchscreen or other device. Additional components which can be included in user device 200 or in housing 201 are further described above with reference to FIG. 1. The housing can further be of an appropriate thickness to include the components described in FIG. 1. Strap 202 can be a strap to hold the user device on a user, such as one made from metal, leather, cloth, or other material. User device 200 can contain a health sensor 140 to perform health sensing functions.

The user device 200 may detect, using one or more sensors, whether it is appropriately secured to the user. For example, where the device 200 is a smartwatch, it may detect whether it is being worn too loosely, too tightly, too high on the user's arm, backwards, etc. A notification on user device 200 can be output to indicate to the user that the device should be adjusted or tightened to give an accurate PPG or other health reading. For example, the notification may be a visual notification, such as on display 203 of the user device while in other examples, according to the capabilities of the user device, other notifications can be given such as through a vibration, an audio alert, a beep, a flash, or other notification.

Although a smartwatch is illustrated as user device 200, user device 200 can take on a variety of forms. For example, user device 200 can be a fitness tracker, an earbud, headphone, arm band, smart glasses, smart clothing, or other wearable electronic device, a ring, a bangle, an anklet, necklace, or other piece of jewelry.

FIG. 2B illustrates an example schematic and cross sectional view of a device 200 placed on top of skin 250 of user 299. While some components are shown, such as user interface 194, it should be understood that the device 200 may include any number of additional components, such as those discussed above in connection with FIG. 1, to provide the suggestions of how tightly the device should be worn.

Sensor(s) 211 may be sensors configured to operate as an “internal” temperature sensor, to provide or estimate the heat generated from or temperature within user device 200. In some examples, “internal” can refer to the configuration of sensor(s) 211 intended to detect the internal temperature of user device 200. Sensor 211 may be made of one or more sensors, transistors, or temperature detection devices, which can be located at various locations within user device 200. In some examples, sensor(s) 211 may be considered to be high confidence sensors and used to increase the accuracy of analysis of information obtained from other sensors by providing a calibration value or offset to other sensors.

Sensor(s) 212 may be sensors configured to operate as “external” temperature sensors, such as to measure or estimate the heat generated from skin 250 or from the environment in which user device 200 is placed. In some examples, “external” can refer to the configuration of sensor(s) 212 to be intended to detect temperature external to user device 200. As further explained herein, this information can be used to estimate the temperature and how a PPG may be degraded due to a colder temperature. In some examples, sensor(s) 212 can be thermally coupled to either the back housing 241 of a user device or via a thermally conductive pad or cutout in a back housing 241 of user device 200.

Sensor(s) 213 may be configured as ambient light sensors. Sensor(s) 213 can thus detect the amount of ambient light entering into the user device at a point where the housing of the device is intended to be secured against the user's body. As further explained below, such detection of ambient light can be used to determine a “tightness” of user device 200 on user 299.

Sensors(s) 214 may be sensors which detect motion of a user, or “secondary” motion of a user device, such as bobbing of user device 200 on user 299's wrist when not tightly secured. Such sensors may include, for example, accelerometers or other motion sensors. The “bobbing” motion may be detected based on a pattern of the detected motion signals. For example, the detected signals may be compared to a library of stored signal patterns to determine whether the signals correspond to a saved pattern indicating bobbing. In some examples, the degree or amount of bobbing can be based on a machine learning model which can be trained with training data to detect, evaluate, and/or rank the probability that the user device, signals obtained from the user device, or other motion signals, is in a “bobbing” condition.

Also illustrated in FIG. 2B is skin 250, with a hypodermis layer 251, a dermis layer 252, and an epidermis layer 253. Epidermis layer 253 is a thinner layer of skin and can permit light to pass through it. The skin contains veins and arteries, such as vein 260 and artery 270. Light generated from user device 200 can be emitted to skin 250. The light emitted can travel through the epidermis layer 253, the dermis layer 252, and be reflected from the veins and arteries within the skin, such as vein 260 or artery 270, and then be reflected back to health sensor(s) 140, such as for example, PPG modules or photodetectors. Light that hits skin 250 reflects off the various layers within the skin depending on the incident angle of the light. The light that hits the skin at shallow angles reflects off the top layer or epidermis layer 253. This reflected light contains little or no heartbeat information as it does not interact with arteries. Light that hits the skin at steeper angles penetrates the top layer of the skin to enter into other layers, such as the hypodermis layer 251 or the dermis layer 252, which contain a strong concentration of veins and arteries that carry blood, such as vein 260 and artery 270. Light that reflects off these layers carries the heartbeat signal and is useful for the purpose of PPG. Variations in the light transmitted to the photodetector can be used to determine various aspects of a cardiovascular system, such as the heart rate, pulse, oxygen saturation in the blood, or other health-related information. In some examples, a wave form can be derived from the continuous or near-continuous monitoring of light received by health sensor(s) 141. As explained above, during colder conditions, a smaller useful signal is obtained from skin 250 of the user due to constriction of the blood vessels, such as vein 260 and artery 270. Further, during an improper fit or a fit which is too loose, a larger amount of ambient light may enter into the user device and decrease the signal to noise ratio for PPG related data.

FIG. 2C illustrates communication between two user devices, user device 200 and user device 290 worn by user 299. In this instance, user device 290 is an earbud, though in other examples the user devices 200, 290 may be any other type of electronic device containing a health sensor, such as health sensor 140. In some examples, communication between the devices can be used to more accurately obtain a signal or estimate an outside condition. For example, if user device 290 is a headphone, an external temperature sensor included therein can provide an additional data point or signal to estimate the external or environmental temperature. In some examples, an alert or notification generated by one device, such as user device 200, can also be displayed or provided to another user device.

As explained below, the following techniques or methods can be used to provide actionable suggestions or notifications to a user to improve health sensor or PPG signals or readings. Data obtained from the sensors described above, such as temperature, motion, and PPG signals, along with confidence or error values for PPG signals, can be used to determine whether a user device can be tightened to improve signal quality through a trained machine learning model or other algorithm.

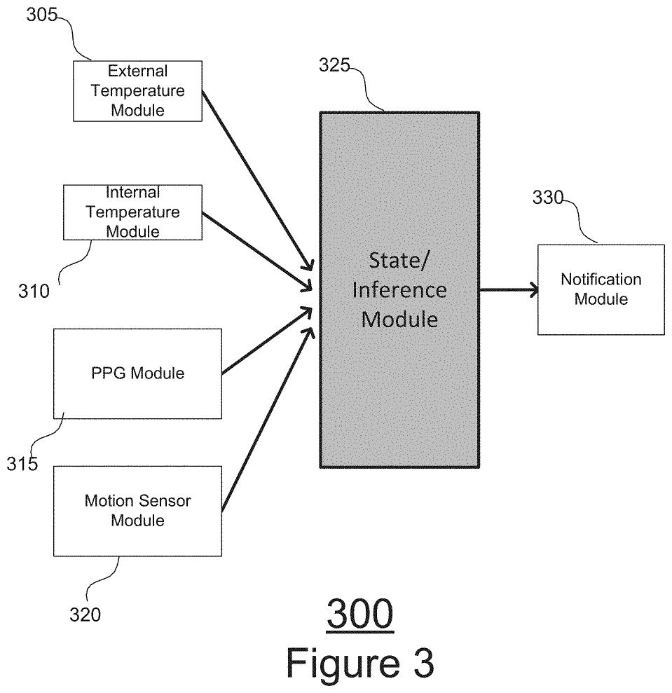

FIG. 3 illustrates an example schematic view of computational architecture 300 used in the device. Architecture 300 can be used to determine whether a user device can be tightened to improve signals being obtained by the user device and to provide notifications to the user to tighten or adjust the user device. FIG. 3 illustrates how multiple inputs, obtained directly from the sensors described herein, or after analysis of data obtained from the sensors, can be used to infer states in which a user device is being used and provide suggestions to a user. The suggestions can be actionable and when implemented, improve a PPG or other health signal. Although an example number of modules are shown, additional configurations, modules, or variations of architecture 300 are possible. For instance, a particular variation may be chosen based on computational efficiency or power concerns in a particular device.

Illustrated in FIG. 3 is external temperature module 305. “External” need not refer to the sensor itself being outside of a user device but rather can refer to a measurement being focused on a user's skin or the ambient air temperature. As one example, temperature module 305 can obtain an estimated or measured temperature from data obtained by sensor(s) 141 and/or sensor(s) 212 after analysis or processing through electronics 199. In some examples, external temperature modules can receive external temperatures from multiple sensors and discard temperatures with low confidence values, outliers, or choose to provide to module 325 only the most likely or temperature with the highest confidence values. The external temperature data can also provide an indication of the temperature ranges at which PPG data is relatively more accurate, such as warmer temperatures.

Internal temperature module 310 can be similar to external temperature module 305. Internal temperature module 305 can obtain an estimated or measured temperature from data obtained by sensor(s) 141 and/or sensor(s) 211 after analysis or processing through electronics 199. Internal temperature module 310 can also analyze multiple temperature values or determine which values to provide for analysis by module 325.

Samples of internal temperatures can also be used for calibration to offset external temperature data. If there is a high power “aggressor” or application running on the user device, which can lead to heat from processing components, an offset may be applied to the external temperature values as this internal heat may cause the external values to show up as higher than the true values. This correlation and a proper offset can be empirically determined in a laboratory for a range of values and stored in memory of a user device.

PPG module 315 can analyze PPG data and/or include PPG algorithms, such as those described herein, and provide health data, such as heart rate. In some examples, a “raw” or unprocessed signal being obtained from one or more PPG sensors or PPG modules can be analyzed using a PPG algorithm to output an estimated heart rate value. The PPG algorithm can also be a machine learning based model. The model can have an associated confidence value, such as for example, values on a 0-100 scale, where 0 indicates a low confidence value and 100 indicates a high confidence value. In some examples, PPG signals with low confidence values or estimated metrics from PPG signals, such as heart rate, can be discarded. In some examples, the confidence value from the PPG signals can also be used in evaluating the state of the user device through the methods and machine learning modules described herein.

Motion sensor input 320 can obtain or analyze information from sensors related to the motion of a user device, such as sensor(s) 141 and sensor(s) 214. At this module, the obtained motion sensor data can be used to understand how much a housing for a user device is moving around versus a baseline or expected movement of the user device. In other examples, motion data can directly be obtained at this module and provided to module 325. As one example, with a looser strap tightness, it can be expected that the user device housing will move around more when the user is engaged in physical activity such as running. Motion sensor data can provide an indication of acceptable degrees of motion as at the correct tightness, there may be less motion resulting in better PPG measurements.

In some examples, information can be “vectorized” before being sent to or analyzed by module 325 which is further described below. The outputs of various modules described above can be vectorized using their numerical values. The numerical values obtained can be mapped onto the same scale. In some examples, a vector can be created or specified by a time value, such as the time at which the specific information forming the numerical values are being mapped into a vector. In this manner, each vector can correspond to or be uniquely related to a specific time value. Although 4 modules are described above, which can generate a 4-dimensional vector, any vector with “N” dimensions can be generated using “N” or more inputs. Although the prior description is provided as a vector, a person of skill in the art will appreciate that a n-tuple or other mathematical formulation of the data can be used.

Module 325 can be a state or inference module which can be used to determine if the user device is properly secured to a user or if the fit and tightness of the user device can be improved.

In some examples, module 325 can contain one or more machine learning models. In some examples the machine learning models can take as inputs external temperature data, internal temperature data, PPG data, and motion sensor data, and provide as an output one or more states. The output states can be related to the dynamic tightness suggestions of the user device.

In some examples, the one or more machine learning models can be trained in laboratory or other controlled settings where the tightness of fit and temperature are known on a set of input data, such as {external temperature data, internal temperature data, PPG data, and motion sensor data} and a set of output data, such as states {0, 1, 2, . . . , n}. In other examples, the output data can be a range, such as 0-100, where “0” can indicate that the tightness of a user device is the worst and “100” can indicate that the tightness is proper and additional tightness would not improve signals being measured or obtained. While the input and output data here are provided as examples, other input and output data can be used for training. For example, metrics derived from the PPG optical inputs, motion sensor data, or temperature data.

In some examples, module 325 can implement density based clustering by using specific test cases where a “bad” or improper fit is present and query a vector with a similar signal or signature (e.g. within a specific error range) to look at the density of the vector space around the specific vector which is “close” or similar to the queried vector. Clusters which are the densest will likely indicate that the factors are related as the sub-components are similar resulting in repeated instances of the vectors appearing in the N-dimensional space. In some examples, a certain threshold, such as measured by a percentage or ratio, must be met before a cluster is considered to be detected or valid, or used as a trigger for a certain condition. In some examples, additional threshold values can be added, such as the size of the cluster, the relative size of the clusters, or a global optimization of the largest or most impactful k-clusters. These clusters are thus trained or evaluated from a clustering-based machine learning model.

In some examples, three primary vectors or points of data, such as (i) PPG accuracy or PPG error, (ii) motion sensor data, and (iii) external temperature can be clustered together using any clustering algorithm.

In some examples, module 325 can contain an implementation which does not require a trained machine learning model but can use an alternative implementation which may be computationally less intensive. In this example implementation or mode, module 325 can determine if a set of data or multiple sets/samples of PPG data have a low confidence value, such as below a predetermined value, simultaneously sample from external and/or internal temperature sensor(s) whether to compensate or adjust for excessive heat from inside the user device, and when the external temperature sensor indicates that the temperature is below a threshold set by a predetermined value, to indicate that the user should tighten a user device or move it higher on his or her wrist. In some examples, the threshold for the external temperature sensor can be determined in a controlled environment, such as a laboratory study, by studying and analyzing known metrics obtained from a test group. In some examples, the threshold for the external temperature sensor can vary based on the confidence value obtained from the PPG data.

Module 325 can output a state to notification module 330. For instance, module 325 can encode a state as a numerical value, which can be interpreted by notification module 330.

Notification module 330 can provide a user notification related to an actionable indicator upon a model or algorithm determining that the fit of a user device can be improved. In some examples, the notification can be interactive and updateable after the user implements the action indicated, such as tightening a strap. In some examples, the notification can be more granular. For instance, if a type of user device or the type of strap being used is known and provided to module 325, the output from notification 325 and in turn the notification can indicate which position to secure the strap in for a better signal from the user device. For example, depending on the state output, different messages can be output by notification module 330, such as those illustrated in Table 1.

TABLE 1

| State | Example Message |
|---|---|
| 1 | “Please tighten your watchband to ensure high heart rate sensor |
| | accuracy. High motion activities require the watch to be fit |
| | more securely on wrist.” |
| 2 | “Please tighten your watchband to ensure high heart rate sensor |
| | accuracy. Colder temperatures cause blood vessels to constrict, |
| | and watch must be fit more securely on wrist.” |
| 1 + 2 | “Please tighten your watchband to ensure high heart rate sensor |
| | accuracy. High motion activities and cold temperatures require |
| | the watch to be fit more securely on wrist.” |

In other examples, the notification can be pre-emptive based on other conditions. For instance, if a user loosens his or her user device when sleeping and habitually takes a run upon waking up, such as at a fixed time, the notification can indicate before the run starts that the user may desire to tighten the user device around his or her person. Additional examples of high motion activities which can degrade the signal include high motion sports with fast movement or arm movement, such as tennis or high intensity interval training workouts. In other examples, swimming or detection of water can also be included in generating notifications. The presence of water can interfere with a PPG signal due to the temperature of the water constricting blood vessels as well as due to light refractions from water being present between the user device and the user.

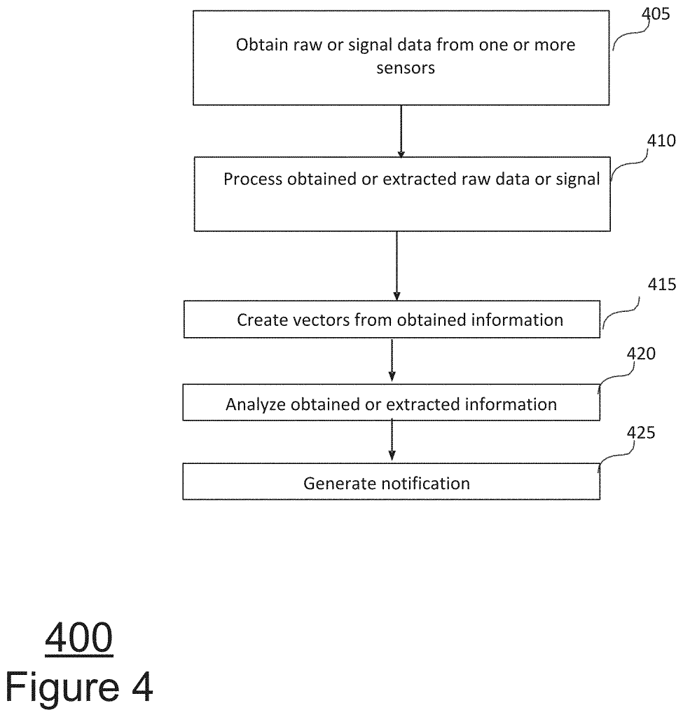

In some examples, notification module 355 can provide a notification upon determining that the data provided indicates “looseness” or the potential for fit to be improved above a predetermined threshold and not provide a notification if the fit or obtained signal would not improve beyond a predetermined threshold. Notification module 355 can perform operations described herein to generate a notification, including those described with respect to FIG. 4.

In some examples, notification module 355 can provide a notification upon determining that the data provided indicates stress above a predetermined threshold. Notification module 355 can perform operations described herein to generate a notification, including those described with respect to FIG. 4.

In some examples, the model and/or module components described with respect to FIG. 3 can be updated based on new information. In some examples, one or more of the following techniques can be used as part of the disclosed technology.

In some examples, probabilistic methods can be used. For example, a gaussian mixture model can be used. Gaussian mixture models are a probabilistic model for representing normally distributed subpopulations within an overall population. In a Gaussian mixture model, it is not required that an observed set of data should characterize or state which subpopulation a particular observation within the distribution belongs to.

Example machine learning techniques which can be used include the following. In some examples, a mix of supervised learning techniques and unsupervised learning techniques can be used.

In some examples, clustering methods can be used to cluster inputs/Clustering methods can be used in real time to classify and match models or groups of models with particular criteria. Clustering can be an unsupervised machine learning technique in which the algorithm can define the output. One example clustering method is “K_Means” where K represents the number of clusters that the user can choose to create. Various techniques exist for choosing the value of K, such as for example, the elbow method.

Some other examples of techniques include dimensionality reduction. Dimensionality reduction can be used to remove the amount of information which is least impactful or statistically least significant. In networks, where a large amount of data is generated, and many types of data can be observed, dimensionality reduction can be used in conjunction with any of the techniques described herein. One example dimensionality reduction method is principle component analysis (PCA). PCA can be used to reduce the dimensions or number of variables of a “space” by finding new vectors which can maximize the linear variation of the data. PCA allows the amount of information lost to also be observed and for adjustments in the new vectors chosen to be made. Another example technique is t-Stochastic Neighbor Embedding (t-SNE).

Ensemble methods can be used, which primarily use the idea of combining several predictive models, which can be supervised ML or unsupervised ML to get higher quality predictions than each of the models could provide on their own. As one example, random forest algorithms

Neural networks and deep learning techniques can also be used for the techniques described above. Neural networks generally attempt to replicate the behavior of biological brains in turning connections between an input and output “on” or “off” in an attempt to maximize a chosen objective.

FIG. 4 illustrates method 400 related to aspects of the disclosed technology.

At block 405, information can be obtained from one or more sensors. The obtained information or data can include data obtained from any of the sensors described herein. In some examples, information can be sampled at different rates by different sensors and collected or combined over a time period to create a sample size for the data. In some examples, data within a particular sample size can be analyzed as described below. In other examples, data in each sample size

At block 410, information obtained at block 405 can be analyzed or processed. In some examples, modules, techniques, classifiers, or models described with respect to FIG. 3 can be used for analysis. In some examples, the information can be analyzed or transformed to output a numerical value related to the obtained or extracted information.

At block 415, the information obtained, transformed, or analyzed in blocks 405-410 can be vectorized.

At block 420, analysis of the obtained or transformed data can be performed using the techniques described herein. In some examples, density based clustering or other clustering techniques can be performed or used to identify clusters or features within data. Clustering is a method or technique in which clusters can be identified within a set of data. Clusters can be separated from sparser noise surrounding the identified clusters. These clusters can be saved or extracted for classifying future or new data. Any clustering technique can be used within block 430.

In some examples, DBSCAN or defined distance scanning is a technique which can use a specified search distance to identify potential clusters. Other clustering technique variations can include self-adjusting scanning or HDB SCAN techniques. This range can vary the search distance to allow a range of distances to be used when separating or identifying clusters of varying densities from sparser noise. Multi-scale or OPTICS methods or algorithms can be used. OPTICS algorithms use the distance between neighboring features to determine a reachability plot, which can then be used to separate clusters. The algorithm can be more computationally intensive but provides flexibility in fine-tuning clusters. Other clustering techniques can include machine learning based clustering techniques. In some examples, a vector can be generated from the identification techniques to represent a state of the wearable device.

At block 425, a notification is output when it is evaluated or determined that the tightness of fit can be improved. In some examples, the notification can be based on the vector output from block 420. An obtained or constructed vector, such as one constructed from the obtained sampled data, can be analyzed using a proximity approach to see if the vector is close to vectors which are closely clustered and meet a threshold of the user device being incorrectly or too loosely attached.

Additional operations can be performed in conjunction with or substituted in method 400. For instance, in some examples, one or more models obtained can be updated. In some examples, the models can be updated through models updated using federated learning (FL) techniques. FL can safeguard privacy by eliminating the requirement of needing specific, individual, or sensitive data in training a global model by using securely encoded model updates instead of the sensitive data. In this manner, federal learning can mitigate privacy risks which can be present in creating machine learning models. Further, FL can allow for a reduced computational cost in training models. FL techniques can enable models to be trained on individual devices and only provide updates to the model periodically with the global model. The global model can be retrained based on the provided updates.

While the above described techniques have been described with respect to a user device which is a wearable device, such as a smartwatch, the same techniques can be used for detecting fit and suggesting improved fit of other devices. For instance, if headphones or earbuds are becoming loose due to physical activity by a user, and a PPG or other health signal is being degraded, the same techniques can cause an audible alert to be sent to the user via the headphones or in-ear devices, such as ear buds. The actionable notification might indicate that the earbuds are slipping from within the ear canal and should be reinserted or pressed in.

While this disclosure contains many specific implementation details, these should not be construed as limitations on the scope of what may be claimed, but rather as descriptions of features specific to particular implementations. Certain features that are described in this specification in the context of separate implementations may also be implemented in combination in a single implementation. Conversely, various features that are described in the context of a single implementation may also be implemented in multiple implementations separately or in any suitable sub-combination. Moreover, although features may be described above as acting in certain combinations and even initially claimed as such, one or more features from a claimed combination may in some cases be excised from the combination, and the claimed combination may be directed to a sub-combination or variation of a sub-combination.

Similarly, while operations are depicted in the drawings in a particular order, this should not be understood as requiring that such operations be performed in the particular order shown or in sequential order, or that all illustrated operations be performed, to achieve desirable results. In certain circumstances, multitasking and parallel processing may be advantageous.

References to “or” may be construed as inclusive so that any terms described using “or” may indicate any of a single, more than one, and all of the described terms. The labels “first,” “second,” “third,” and so forth are not necessarily meant to indicate an ordering and are generally used merely to distinguish between like or similar items or elements.

Various modifications to the implementations described in this disclosure may be readily apparent to those skilled in the art, and the generic principles defined herein may be applied to other implementations without departing from the spirit or scope of this disclosure. Thus, the claims are not intended to be limited to the implementations shown herein, but are to be accorded the widest scope consistent with this disclosure, the principles and the novel features disclosed herein.

1. A method for providing information related to fit of a wearable device, the method comprising:
receiving external temperature sensor data from one or more sensors configured to obtain an external temperature of the wearable device
receiving health data from one or more health sensors of the wearable device;
analyzing, using a trained machine learning model, metrics related to the external temperature sensor data and the health data, to obtain an output;
determining, from the output, whether to suggest adjusting the fit of the wearable device; and
generating a notification based on the determining.

2. The method of claim 1, comprising receiving internal temperature sensor data from one or more sensors configured to obtain temperature from inside the wearable device

3. The method of claim 2 wherein a temperature offset is applied to the external temperature sensor data based on the measured internal temperature sensor data.

4. The method of claim 1 further comprising detecting a change in the fit upon a user taking an action on the user device.

5. The method of claim 4 further comprising comparing a second output to the first output, wherein the second output is obtained by analyzing, using a trained machine learning model, metrics obtained after detection of a change in the fit.

6. The method of claim 5 further comprising updating or removing the notification when the second output sufficiently differs from the first output.

7. The method of claim 1 wherein the trained model is a clustering model.

8. The method of claim 1 wherein the health sensor is a PPG sensor.

9. The method of claim 1 wherein the one or more sensors comprise a gyroscope or accelerometer.

10. The method of claim 1 further comprising receiving motion sensor data from one or more motion sensors of the wearable device and wherein the analyzing analyzes metrics related to the motion sensor data.

11. The method of claim 10 wherein the motion sensor data is analyzed to determine a bobbing motion of the wearable device.

12. The method of claim 1 wherein additional external temperature data is obtained from a second wearable device

13. The method of claim 1 wherein health data is raw or processed data from which photoplethysmography can be performed.

14. A wearable device, comprising:
a communications interface;
a display; and
one or more computing devices coupled to one or more memory devices, the one or more memory devices containing instructions that cause the one or more computing devices to:
receive external temperature sensor data from one or more sensors configured to obtain an external temperature of the wearable device
receive health data from one or more health sensors of the wearable device;
receive motion sensor data from one or more motion sensors of the wearable device;
analyze, using a trained machine learning model, metrics related to the external temperature sensor data and, the health data, and the motion sensor data, to obtain an output;
determine, from the output, whether to suggest adjusting the fit of the wearable device; and
generate a notification based on the determining.

15. The wearable device of claim 14 further comprising the instructions configured to receive internal temperature sensor data from one or more sensors configured to obtain temperature from inside the wearable device

16. The wearable device of claim 15 wherein a temperature offset is applied to the external temperature sensor data based on the measured internal temperature sensor data.

17. The wearable device of claim 14 further comprising the instructions configured to detect a change in the fit upon a user taking an action on the user device.

18. The wearable device of claim 17 further comprising the instructions configured to compare a second output to the first output, wherein the second output is obtained by analyzing, using a trained machine learning model, metrics obtained after detection of a change in the fit.

19. The method of claim 14 further comprising the instructions configured to receive motion sensor data from one or more motion sensors of the wearable device and wherein the analyzing analyzes metrics related to the motion sensor data.

20. A non-transient computer readable medium containing program instructions, the instructions when executed perform the steps of:
receiving external temperature sensor data from one or more sensors configured to obtain an external temperature of a wearable device
receiving health data from one or more health sensors of the wearable device;
analyzing, using a trained machine learning model, metrics related to the external temperature sensor data and the health data, to obtain an output;
determining, from the output, whether to suggest adjusting the fit of the wearable device; and
generating a notification based on the determining.
